(19) United States
(12) Patent Application Publication
(10) Pub. No.: US 2026/0306956 A1
(43) Pub. Date: Oct. 8, 2026

(54) PERSISTENT EMOTIONAL CORE MODULE FOR MULTI-HOUSING INTERACTIVE COMPANIONS

(71) Applicant: AIfection LLC, Cumming, GA (US)

(72) Inventor: Nhu Nguyen, Cumming, GA (US)

(73) Assignee: AIfection LLC, Cumming, GA (US)

(21) Appl. No.: 19/638,241

(22) Filed: Apr. 3, 2026

Related U.S. Application Data
(60) Provisional application No. 63/783,980, filed on Apr. 5, 2025.

(51) Int. Cl. A63H 3/00; A61B 5/00; A61B 5/16; A61M 21/02; A63H 3/28; G06N 3/008; G10L 17/02; G10L 25/63; H05K 5/02

(52) U.S. Cl. CPC A63H 3/003; A61B 5/165; A61B 5/4803; A61B 5/4836; A61B 5/6896; A61M 21/02; A63H 3/006; A63H 3/28; G06N 3/008; G10L 17/02; G10L 25/63; H05K 5/026; A63H 2200/00

(57) ABSTRACT

A removable, heart-shaped electronic module, referred to as a core, stores and processes personalized emotional interaction data for a user and is physically transferable between multiple interactive companion housings. The module maintains persistent emotional identity, voice recognition, adaptive comfort learning, mood pattern tracking, and personalized routine storage across transfers. Each companion housing provides a standardized docking interface with passive audio, visual, and haptic components, while all processing and memory reside on the removable module. A hybrid three-layer connectivity architecture enables local-first operation, optional Bluetooth connection to a companion application, and encrypted cloud backup and restore, with an age-tiered privacy consent system that automatically adjusts data access and monitoring based on user age or care status.

### CROSS-REFERENCETORELATEDAPPLICATIONS

This application claims the benefit of U.S. Provisional Patent Application No. 63/783,980, entitled “AI-Enhanced Modular Interactive Plush Toy System,” filed Apr. 5, 2025, by inventor Mrs. Nhu Thi Nguyen, the entirety of which is hereby incorporated by reference.

### BACKGROUND

### Field of The Invention

The present invention relates to interactive electronic companion systems, and more particularly to a removable, heart‑shaped electronic module that stores a user’s persistent emotional profile and personalized interaction data while transferring among multiple companion housings. The invention further relates to companion housings with standardized docking interfaces and passive audio, visual, and haptic components, and to a connectivity architecture supporting local‑first operation, optional Bluetooth links to an application, and encrypted cloud backup with age‑tiered privacy controls.

### Existing Technologies and Challenges

Various electronic toys, smart speakers, and social robots provide interactive audio, visual, and haptic experiences, and some employ cloud-connected artificial intelligence to personalize responses based on user input. However, these systems typically bind personalization data and interaction history to a single device form factor, such that any replacement device or alternative character housing requires new training, separate configuration, or fragmented accounts for the same user. Existing platforms also often depend on continuous or frequent internet connectivity to deliver core functionality, creating reliability, latency, and privacy concerns in network-limited or sensitive environments such as children’s bedrooms, clinical settings, or elder care facilities.

Current solutions further lack a unified mechanism for physically transferring a user’s emotional identity, comfort preferences, and longitudinal mood profile between multiple soft-body companions or other housings in a way that preserves continuity of experience and traceability of transfers. At the same time, many consumer and clinical products provide only coarse or static privacy controls, rather than dynamic, age-tiered consent frameworks that can automatically adapt monitoring visibility, data retention, and third-party data sharing as a user matures or transitions between independent and caregiver-managed states. Accordingly, there is a need for a removable electronic module that functions as a persistent emotional core across multiple companion housings, operates in a local-first manner with optional layered connectivity, and incorporates age-aware privacy governance to align with diverse regulatory and ethical requirements.

The present invention addresses these challenges by providing such a removable emotional core module and associated companion housings, connectivity architecture, and age-aware privacy and consent mechanisms.

### SUMMARY

In one embodiment, an interactive companion apparatus includes a removable electronic module receivable in any of a plurality of different companion device housings, the removable electronic module comprising a processor, non‑volatile memory storing a persistent emotional profile comprising personalized interaction data for a primary user, and wireless communication circuitry, and the plurality of companion device housings each having a standardized docking interface and one or more output components including at least an audio output element and a light output element, such that, when the removable electronic module is transferred between different ones of the plurality of companion device housings, the processor personalizes interactions with the primary user based on the persistent emotional profile without requiring model retraining or loss of stored emotional history. The persistent emotional profile can include a voice identity signature used to recognize the primary user across different housings and sessions, an adaptive comfort profile that maps multimodal comfort interventions to measured effectiveness and is updated over time, a longitudinal mood profile derived from interaction patterns and optionally synchronized to a companion application, and a plurality of caregiver‑defined routines specifying ordered sequences of audio, light, and haptic outputs that execute in a substantially identical manner regardless of which companion device housing receives the removable electronic module.

In another embodiment, the standardized docking interface of each companion device housing comprises a recessed cavity shaped to receive the removable electronic module, guiding features such as magnetic alignment elements or asymmetric mechanical geometry that bias the removable electronic module into a desired orientation, a plurality of electrical contacts that couple the removable electronic module to the output components, and a retention mechanism such as a bayonet‑style or quarter‑turn interface configured to secure the removable electronic module and provide an audible or tactile indication when secured. Each companion device housing can further include a passive identification element readable by the processor so that, upon insertion of the removable electronic module, the processor logs a transfer event including at least a timestamp and a housing identifier in the non‑volatile memory and updates the persistent emotional profile based at least in part on a history of transfers, and a sensory feedback subsystem controlled by the processor and including the light output element and a haptic actuator to generate synchronized light and tactile patterns, such as calming heartbeat‑style sequences during soothing routines and active engagement sequences during conversational or play interactions, whose pulse rate, intensity, color, and pattern are modulated according to the persistent emotional profile and real‑time emotional state inferences about the primary user.

In still another embodiment, the removable electronic module operates according to a multi‑layer connectivity architecture comprising: a primary layer in which core interaction features execute locally using the persistent emotional profile without a network connection; a secondary layer in which the removable electronic module communicates via a short‑range wireless protocol with a companion application to exchange configuration data, content, and interaction summaries; and a tertiary layer in which the companion application communicates with remote services to perform artificial‑intelligence processing and encrypted backup and restore of at least a portion of the persistent emotional profile while the removable electronic module lacks direct wide‑area network connectivity. In the tertiary layer, the companion application can transmit interaction summaries or audio streams to a cloud‑based artificial‑intelligence service, receive generated responses and route them back to the removable electronic module for playback through the audio output element while enforcing privacy policies, and can maintain an encrypted cloud backup of at least the persistent emotional profile and restore the persistent emotional profile to a replacement removable electronic module following a loss or damage event. An age‑tiered privacy management subsystem associates the persistent emotional profile with an age classification of the primary user, selects and enforces corresponding privacy rulesets over monitoring visibility, data collection scope, transcript retention, and third‑party data sharing while the persistent emotional profile remains associated with the primary user across age classifications, and, upon detecting transitions between age classifications, preserves the persistent emotional profile while modifying which caregivers or gatekeepers may access mood summaries, interaction histories, or other portions of the persistent emotional profile without deleting accumulated emotional data.

### BRIEF DESCRIPTION OF THE FIGURES

The accompanying drawings illustrate various embodiments and along with the description, help explain the principles of the method. It is understood by those skilled in the art that the specific arrangements shown in the drawings are merely examples and should not be considered as limiting the scope of the method or the claims in any way.

FIG. 1: Perspective view of core module

FIG. 2: System-level environment diagram

FIG. 3: Companion housing and docking bay

FIG. 4: Electrical and functional block diagram of core

FIG. 5: Inter-housing portability and transfer logging

FIG. 6: Emotion-responsive sensory feedback subsystem

FIG. 7: Three-layer connectivity and data flow

FIG. 8: Age-tiered privacy governance interface/logic

### DETAILED DESCRIPTION

### System Overview

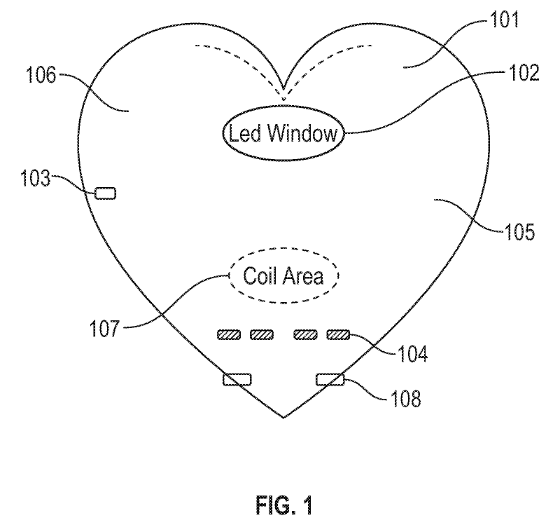

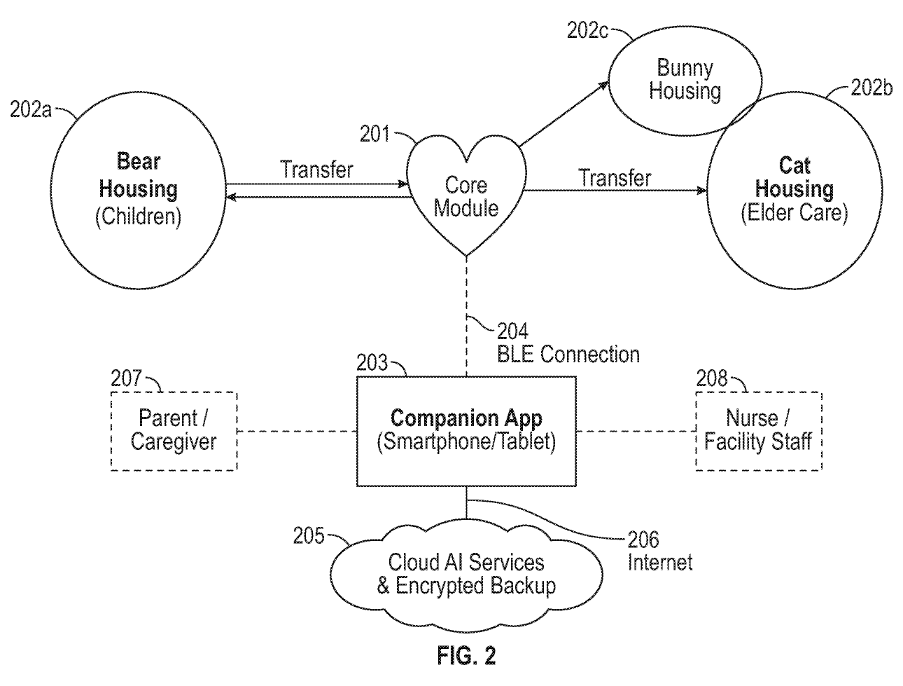

The disclosure provides a modular interactive companion system and method for capturing, storing, transferring, and restoring personalized emotional interaction data across multiple soft‑body companion housings by means of a removable Core module 101. The system enables users including children, adolescents, adults, elderly individuals, and care recipients to form a persistent emotional relationship with a single electronic Core that can be physically moved between different companion housings while maintaining voice identity recognition, adaptive comfort learning, mood pattern tracking, personalized routines, and associated metadata. With reference now to FIGS. 1 and 2, the Core module 101 is configured for insertion into multiple different housings such as a bear housing for children, a cat housing for elder care, and a bunny housing for wellness. Each compatible housing includes a standardized docking interface that cooperates with the Core and provides passive components such as speakers, microphones, light diffusers, and haptic actuators, while all processing, personalization logic, and non‑volatile storage are located on the Core. In this manner, the emotional identity of the user is decoupled from any specific physical character or form factor and is instead bound to the removable Core.

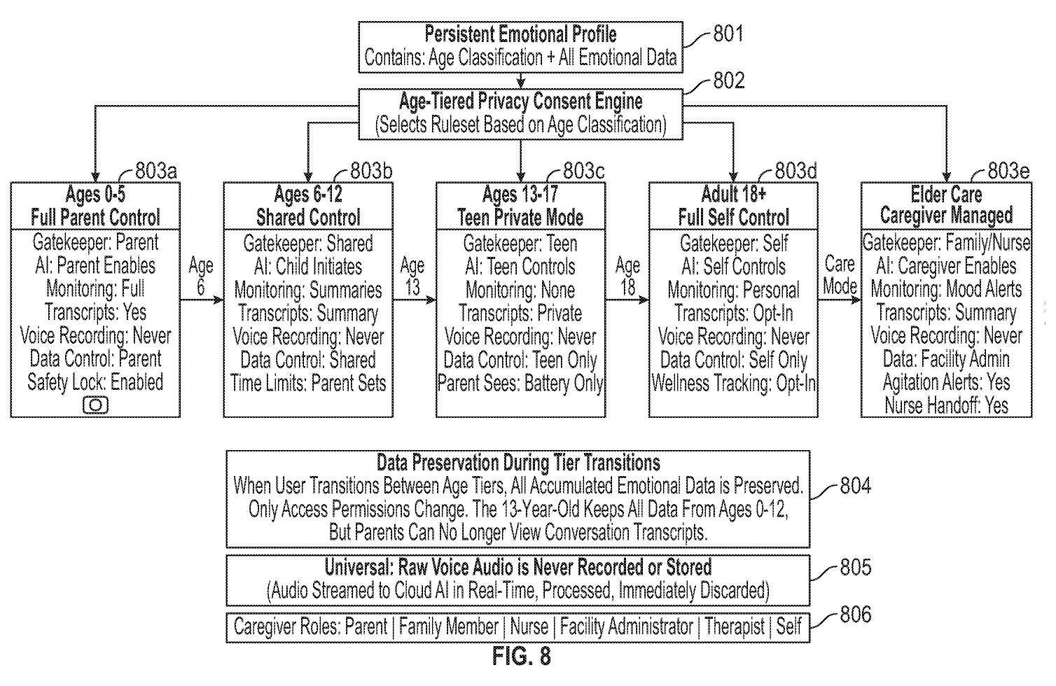

With reference still to FIG. 2, a hybrid connectivity architecture is provided in which the Core supports local operation, a Bluetooth Low Energy connection to a companion application on a smartphone or tablet 203, and optional encrypted cloud services 204 and 205. The Core can operate entirely offline using its local non‑volatile memory so that voice recognition, comfort playback, heartbeat light, and haptic feedback continue to function in the absence of network connectivity. The companion application 203, when present, enables configuration of routines, adjustment of voice and personality settings, visualization of mood trends, caregiver alerts, content downloads, and routing of audio streams toward or from cloud‑based artificial intelligence services. The companion application further serves as a privacy gateway so that the Core never directly connects to the internet 206. An age‑tiered privacy consent framework automatically adapts monitoring and access rules depending on user age or care status such as parental control for young children, shared control for older children, private modes for adolescents, self‑control for adults, and caregiver‑managed modes for elder care, as illustrated in FIG. 8.

### Physical Configuration and Core–Housing Interface

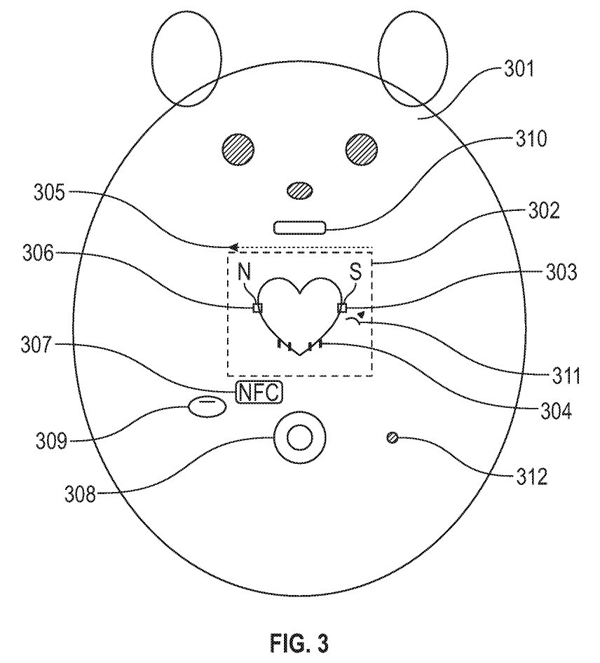

With reference now to FIG. 3, in one embodiment the Core module 101 is implemented as a heart‑shaped or similarly symbolic element that is received within a chest region of a soft companion housing 301. The housing body 301 defines an internal cavity that is aligned with a visible heart region 311 on the front of the housing. An arrangement of magnet poles 302 and an internal LED array 303 are positioned proximate to that region, and additional surface elements such as eyes 310 and a nose 312 may be formed to present an engaging character appearance. The housing may further include fabric light windows and tactile zones 305 through 309 that permit light and vibration from the Core to diffuse through the outer surface in a controlled manner.

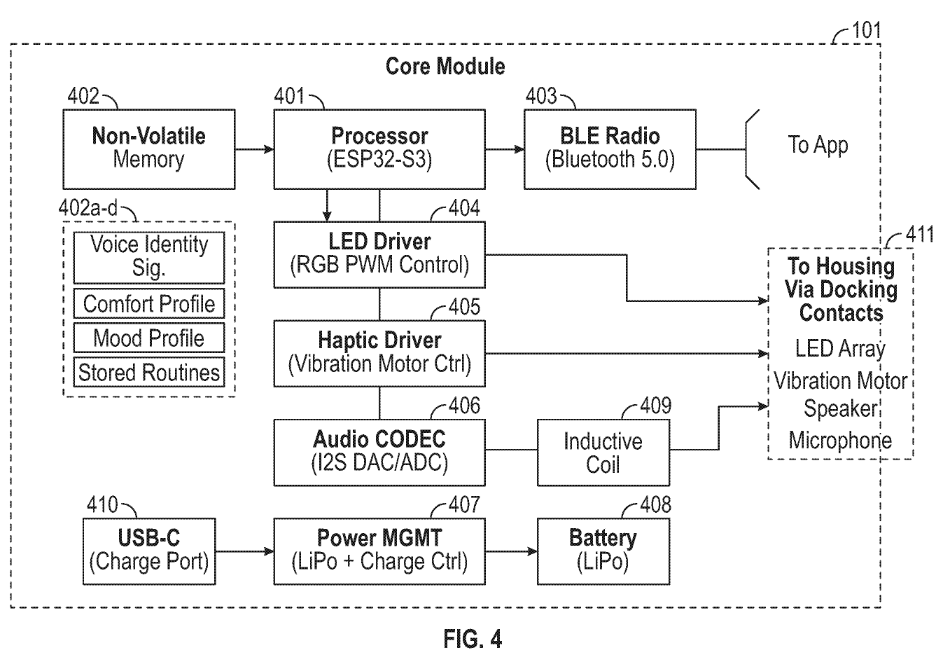

With reference now to FIG. 4, the Core module 101 includes a processor 401 such as a microcontroller, non‑volatile memory 402, a Bluetooth Low Energy radio 403, and a plurality of peripheral driver circuits. The non‑volatile memory 402, which may be organized into subregions 402a through 402d, stores a voice identity signature, an adaptive comfort profile, a mood profile, stored routines, pre‑loaded content, family voice messages, and a transfer history log. A light emitting diode driver 404 provides color and brightness control for an LED array coupled to the housing through docking contacts 411. A haptic driver 405 drives a vibration motor coupled to the housing structure so that haptic energy is transmitted through the body of the housing. An audio codec 406 interfaces with a speaker and a microphone disposed within the housing, while a power management block 407 manages a rechargeable battery 408, an inductive charging coil 409, and a charging port 410 such as a Universal Serial Bus Type C connector. In this configuration, all active electronics and emotional data storage reside on the Core, while the housing provides only passive transducers, fabric, and mechanical structure.

### Emotional Data Architecture Phase

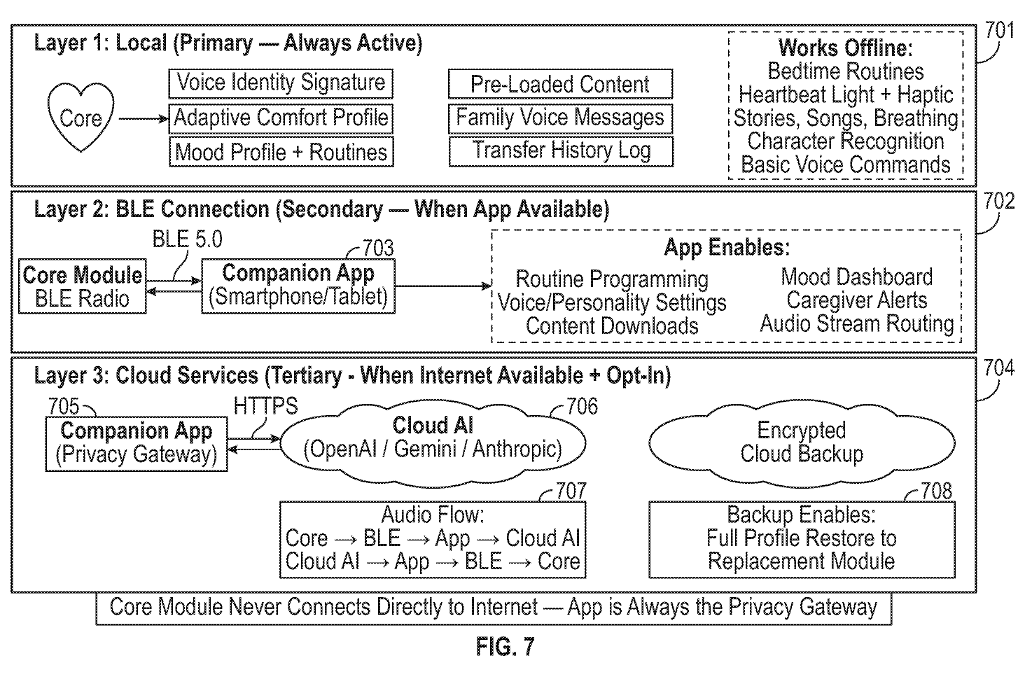

According to an embodiment, the Core implements an emotional data architecture in which multiple categories of personalized emotional information are stored and maintained for a primary user identity. During an initial setup phase, the Core captures voice features of the primary user including pitch, cadence, and tone patterns and derives a persistent voice identity signature that allows recognition of the user across different housings and across multiple sessions. This voice identity signature is stored in the non‑volatile memory 402 and is conceptually represented as a voice identity signature block in FIGS. 4 and 7.

The Core then observes user responses to comfort interventions such as spoken phrases, music, stories, breathing exercises, heartbeat light patterns, and haptic rhythms. Over repeated interactions, the Core builds an adaptive comfort profile that identifies which interventions most reliably calm or support the user under given circumstances. The Core further aggregates interaction metrics describing frequency and duration of use, time of day patterns, inferred emotional tone, and transitions between emotional states in order to construct a longitudinal mood profile. This mood profile can be synchronized with the companion application to support a mood dashboard, caregiver alerts, and clinical or wellness insight as indicated in FIG. 7.

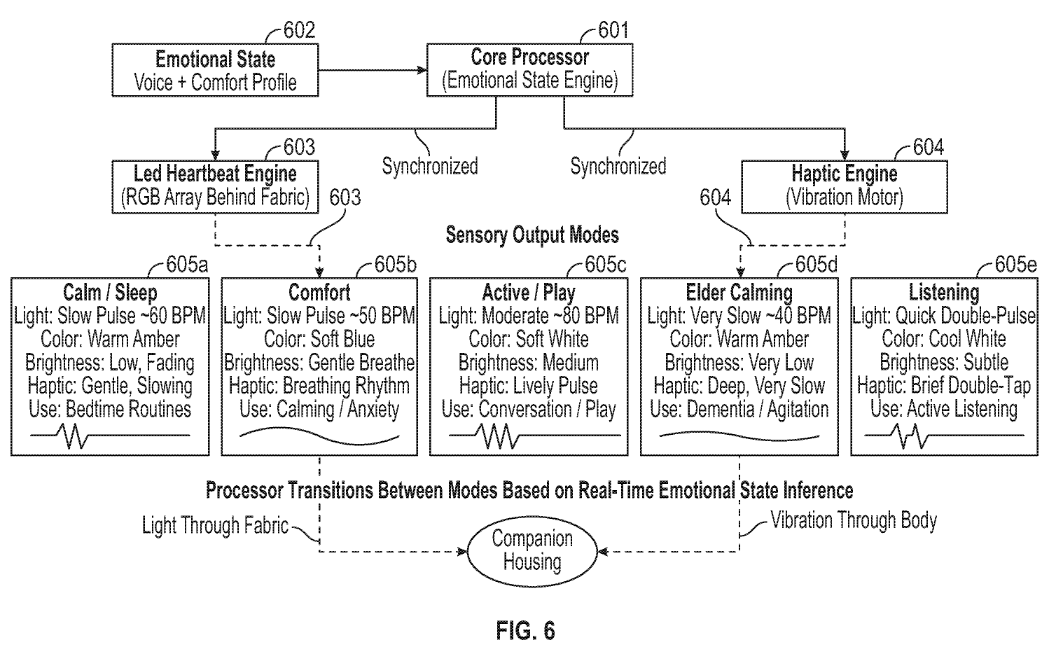

With reference again to FIG. 4, the non‑volatile memory on the Core stores a set of programmed routines that define ordered sequences of audio, visual, and haptic outputs, along with associated timing parameters and triggers. Caregivers or authorized users define these routines through the companion application and then transfer them to the Core, which executes them locally so that behavior remains consistent regardless of the housing in which the Core resides. Routines can reference specific sensory output modes such as calm sleep, comfort, active play, elder calming, or listening, which are described with respect to FIG. 6.

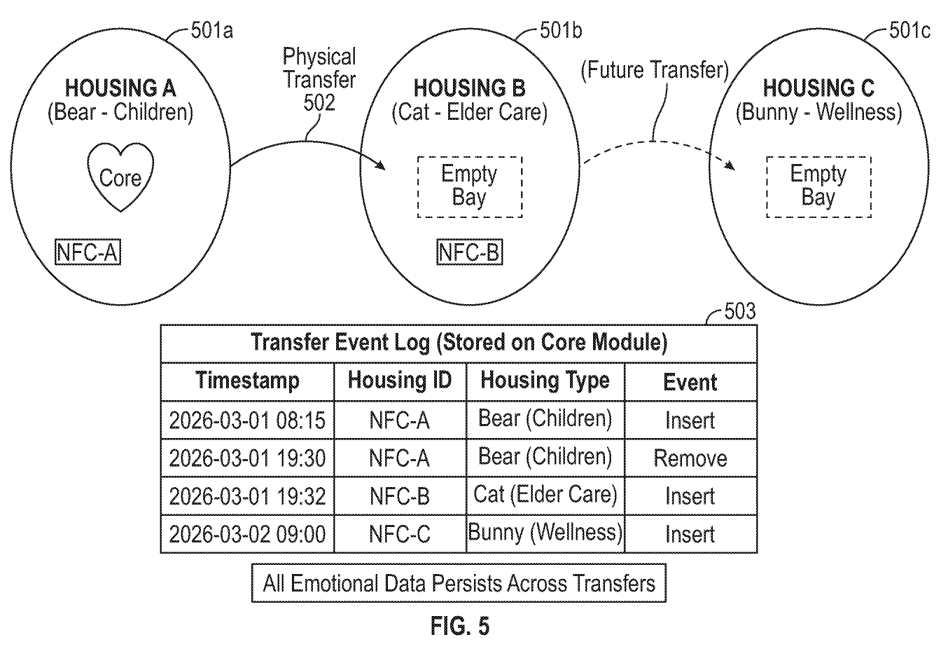

With reference now to FIG. 5, the Core maintains a transfer event log 503 that records each insertion and removal of the Core from a housing. For each event, the log may store a timestamp, a housing identifier such as an identifier read from a near field communication tag, a housing type such as bear for children, cat for elder care, or bunny for wellness, and an event type such as insert or remove. This log allows reconstruction of the physical context in which emotional interactions occurred and supports life‑long traceability of emotional data across multiple characters and product generations.

### Inter‑Housing Portability and Docking Phase

In one embodiment, each interactive companion housing in the system includes a standardized docking bay that receives the Core module 101 and facilitates inter‑housing portability. With reference again to FIGS. 1 and 3, the docking bay is located in a chest region behind a concealed opening so that the Core resides approximately where a heart of a character would be located. The docking bay may incorporate magnetic alignment features corresponding to the magnet poles 302, an asymmetric cavity geometry, or both so that the heart‑shaped Core is guided into a single correct orientation and retained by a mechanical lock such as a quarter‑turn engagement. Electrical contacts within the bay mate with docking contacts on the Core to couple the Core to the passive speaker, microphone, light emitting diodes, and vibration motor that are embedded in the housing.

Each housing incorporates a passive identification element such as a near field communication tag or resistor‑based signature that the Core reads upon insertion in order to determine housing type and log a corresponding transfer event. With continued reference to FIG. 5, housing A is depicted as a bear housing for children associated with an NFC‑A identifier, housing B is depicted as a cat housing for elder care associated with an NFC‑B identifier, and housing C is depicted as a bunny housing for wellness associated with an NFC‑C identifier. The transfer log records insertion and removal events across these housings, while emotional data including voice identity, comfort profile, and mood profile remains on the Core and persists across transfers.

In certain implementations intended for young children or cognitively impaired users, the docking assembly may further include a safety release or recessed latch that requires deliberate manipulation by a caregiver to remove the Core. This reduces risks of choking or tampering while still enabling caregivers to transfer the Core between different housings as needed.

In another embodiment, the same Core is transferred over time between a series of housings that correspond to different life stages and contexts. A child may begin with a brightly colored bear housing, later transition to a more neutral wellness pillow or bunny housing during adolescence, and eventually move to a cat housing optimized for elder care in which the device rests on a lap. In each case, the acoustic and haptic coupling to the body changes, but the Core maintains continuity of emotional identity, comfort routines, and historical interaction data. Additional housings such as seasonal characters or clinical housings used only in supervised facilities may be introduced without modification to the Core architecture so that emotional portability extends over long periods of time.

### Emotion‑Responsive Sensory Feedback Phase

With reference now to FIG. 6, certain embodiments of the system provide an emotion‑responsive sensory feedback subsystem that coordinates light and haptic outputs in response to real‑time emotional state inference. A core processor 601 implements an emotional state engine 602 that evaluates input signals including voice features and interaction patterns. Based on this evaluation, the processor commands a heartbeat light engine 603 and a haptic engine 604 to adopt one of multiple sensory output modes 605a through 605e.

In one mode suitable for calm sleep, the light output provides a slow pulse at approximately sixty beats per minute, with warm amber color and low fading brightness, and the haptic output provides a gentle vibration that slows over time. In another mode intended for comfort and anxiety reduction, the light output provides a slow pulse at approximately fifty beats per minute with a soft blue color and a breathing pattern of brightness, while the haptic output provides a breathing rhythm vibration. In an active play mode, the light output provides a moderate pulse at approximately eighty beats per minute with soft white color and medium brightness, and the haptic output provides a lively pulse. In an elder calming mode, the light output provides a very slow pulse at approximately forty beats per minute with warm amber color and very low brightness, and the haptic output provides a deep and very slow vibration. In a listening mode, the light output provides a quick double pulse with cool white color and subtle brightness, and the haptic output provides a brief double tap.

Light generated by the heartbeat engine 603 passes through the fabric of the housing, while vibrations generated by the haptic engine 604 propagate through the body of the housing, so that a user holding the companion experiences a synchronized pattern of light and tactile sensation that corresponds to an inferred emotional state. The processor transitions between these modes based on changes in the emotional state as inferred in real time.

In certain use cases such as dementia care or trauma recovery, the system combines the sensory patterns with selected audio content including recorded family messages, stories, songs, breathing exercises, or therapeutic soundscapes that are tailored according to the adaptive comfort profile stored on the Core. This multi‑modal combination can reduce agitation, reinforce a sense of safety, and provide predictable transitions for events such as bedtime, medication windows, or therapy sessions. In additional embodiments, caregivers may configure multi‑step rituals in which the Core transitions through a sequence of modes, beginning with a listening mode, passing through comfort and breathing modes, and ending in calm sleep, with the entire sequence stored as a routine in the non‑volatile memory.

### Hybrid Connectivity and Privacy Governance Phase

With reference now to FIG. 7, the system applies a three‑layer connectivity model combined with an age‑tiered privacy governance framework to control data flow and external access. At a primary local layer 701, the Core operates in an offline manner using its non‑volatile memory 402 to store emotional profiles, routines, interaction histories, pre‑loaded content, and transfer logs. At this layer, core features such as bedtime routines, heartbeat light and haptic output, stories, songs, breathing exercises, character recognition, and basic voice commands continue to function at all times.

At a secondary layer 702 and 703, the Core establishes a Bluetooth Low Energy link to a companion application 704 executing on a smartphone or tablet. Through this link, the application enables routine programming, configuration of voice and personality settings, content downloads, mood dashboards, caregiver alerts, and audio stream routing. The companion application also functions as a privacy gateway by mediating all communications between the Core and any networks so that the Core never directly connects to the internet.

At a tertiary layer 704 through 708, when the companion application has network connectivity and when privacy rules allow, the application communicates with cloud services. These services may include artificial intelligence platforms for advanced language processing and emotion analysis and encrypted cloud backup services for storage and restoration of Core data. Audio may flow from the Core through the Bluetooth link to the application and then to cloud artificial intelligence services, and responses may flow back along the reverse path. Encrypted backup of the emotional profile, comfort profile, mood profile, routines, and transfer history allows full profile restoration to a replacement Core module.

With reference now to FIG. 8, an age‑tiered privacy consent engine 802 governs access policies for different user categories and care modes. A persistent emotional profile 801 contains an age classification and associated emotional data. The consent engine selects a ruleset based on the age classification and a care mode indicator 803a through 803e.

In one example mode corresponding to ages zero through five, a parent acts as gatekeeper, monitoring is full, transcripts of interactions are available to the parent, voice recording is never stored as raw audio, data control resides with the parent, and a safety lock feature can be enabled. In another mode corresponding to ages six through twelve, control is shared between the parent and the child, monitoring is provided in summary form, transcripts are summarized, voice recording as raw audio is never stored, data control is shared, and time limits may be set by the parent. In a further mode corresponding to ages thirteen through seventeen, the adolescent acts as gatekeeper, monitoring is disabled, transcripts are private to the adolescent, voice recording as raw audio is never stored, data control resides with the adolescent, and parents may only view limited status such as battery level.

For adults eighteen and older, the user acts as gatekeeper, monitoring is configured for personal use, transcripts are available only on an opt‑in basis, voice recording as raw audio is never stored, data control resides with the user, and wellness tracking may be enabled on an opt‑in basis. In an elder care mode, a family member, nurse, facility administrator, therapist, or similar caregiver acts as gatekeeper, monitoring includes mood alerts, transcripts are provided in summary form, voice recording as raw audio is never stored, facility administrators manage data, agitation alerts are generated, and nurse handoff events are supported. In all modes, raw voice audio is not recorded or stored; instead, audio is streamed to cloud artificial intelligence services in real time when allowed, processed, and then discarded while only derived metadata and emotional features are retained.

FIG. 8 further illustrates data preservation during tier transitions. When the user transitions between age tiers, all accumulated emotional data remains preserved and only access permissions change. For example, when a child reaches age thirteen and moves into the adolescent tier, the user retains all emotional data recorded from ages zero through twelve, while parental access to conversation transcripts is removed. The same principle applies when the user transitions into or out of an elder care mode, thereby maintaining continuity of emotional history while respecting evolving consent and privacy expectations.

### General Remarks

Detailed embodiments of the claimed structures and methods are disclosed herein; however, the disclosed embodiments are illustrative in nature and are not intended to limit the scope of the claimed subject matter. The structures and methods described may be embodied in many different forms and should not be construed as limited to the exemplary embodiments set forth herein. Rather, these exemplary embodiments are provided to ensure that this disclosure is thorough and complete and that it fully conveys the scope of the invention to those skilled in the art.

The terms and words used in the foregoing description and in the claims are not limited to their strict bibliographical meanings but are selected to enable a clear and consistent understanding of the invention. Accordingly, it should be apparent to those skilled in the art that the description of exemplary embodiments is provided for illustration purposes only and not for the purpose of limiting the invention as defined by the appended claims and their equivalents.

It is to be understood that the singular forms “a,” “an,” and “the” include plural referents unless the context clearly dictates otherwise. Thus, reference to a Core module includes reference to one or more such modules unless the context clearly dictates otherwise.

In the context of the present application, a system may comprise a single device or a collection of distributed devices configured to execute one or more embodiments of the methods described herein. For example, a system may include a Core module installed in a single companion housing, a Core module in communication with a companion application, or a collection of Core modules, companion devices, applications, and backend services connected by one or more networks and cooperating to implement at least one embodiment of the described emotional portability platform.

The descriptions of the various embodiments presented herein are not intended to be exhaustive or to limit the invention to the precise forms disclosed. Many modifications and variations will be apparent to those of ordinary skill in the art in view of this disclosure without departing from the scope of the described embodiments. The terminology used herein is selected to explain the principles of the embodiments, the practical applications, and, where applicable, technical improvements over existing technologies, and to enable others of ordinary skill in the art to make and use the embodiments.

1. An interactive companion apparatus comprising:
a removable electronic module receivable in any of a plurality of companion device housings, the removable electronic module comprising a processor, non-volatile memory, and wireless communication circuitry; and
the plurality of companion device housings each having a standardized docking interface and one or more output components including at least an audio output element and a light output element;
wherein the non-volatile memory stores a persistent emotional profile comprising personalized interaction data for a primary user, and the processor is configured, when the removable electronic module is transferred between different ones of the plurality of companion device housings, to personalize interactions with the primary user based on the persistent emotional profile without retraining or loss of stored emotional history.

2. The apparatus of claim 1, wherein the persistent emotional profile comprises a voice identity signature generated from vocal characteristics of the primary user and used by the processor to recognize the primary user across different companion device housings and sessions.

3. The apparatus of claim 1, wherein the persistent emotional profile comprises an adaptive comfort profile that maps comfort interventions including audio content, breathing-style patterns, light patterns, and haptic patterns to measured effectiveness in calming or supporting the primary user, and wherein the processor is configured to update the adaptive comfort profile over time and apply it across the plurality of companion device housings.

4. The apparatus of claim 1, wherein the persistent emotional profile comprises a longitudinal mood profile representing at least interaction frequency, session duration, time-of-day patterns, and emotional tone over time, the longitudinal mood profile being maintained in the non-volatile memory across transfers of the removable electronic module and optionally synchronized to a companion application on a separate computing device.

5. The apparatus of claim 1, wherein the persistent emotional profile further comprises a plurality of caregiver-defined routines, each specifying an ordered sequence of audio, light, and haptic outputs, and wherein the processor is configured to execute the routines in a substantially identical manner regardless of which of the plurality of companion device housings receives the removable electronic module.

6. The apparatus of claim 1, wherein the standardized docking interface of each of the plurality of companion device housings comprises a recessed cavity shaped to receive the removable electronic module, guiding features configured to bias the removable electronic module into a desired orientation, a plurality of electrical contacts configured to couple the removable electronic module to the output components, and a retention mechanism configured to secure the removable electronic module within the recessed cavity.

7. The apparatus of claim 6, wherein the guiding features comprise at least one of magnetic alignment elements or asymmetric mechanical geometry that prevents full insertion of the removable electronic module in an incorrect orientation.

8. The apparatus of claim 6, wherein the retention mechanism comprises a bayonet-style or quarter-turn interface that generates at least one of an audible or tactile indication when the removable electronic module is secured and includes a recessed release control positioned to require deliberate actuation by an adult user.

9. The apparatus of claim 1, wherein each of the plurality of companion device housings further comprises a passive identification element, and wherein the processor is configured, upon insertion of the removable electronic module into a given companion device housing, to read the passive identification element, log a transfer event including at least a timestamp and a housing identifier in the non-volatile memory, and update the persistent emotional profile based at least in part on a history of transfers.

10. The apparatus of claim 1, further comprising a sensory feedback subsystem controlled by the processor and comprising the light output element and a haptic actuator, wherein the light output element is configured to emit pulsed illumination through an exterior portion of a companion device housing, the haptic actuator is configured to generate tactile pulses, and the processor is configured to modulate at least one of pulse rate, intensity, color, or pattern of the pulsed illumination and tactile pulses based on the persistent emotional profile and real-time emotional state inferences about the primary user.

11. The apparatus of claim 10, wherein the processor is configured to generate at least two different synchronized light and haptic patterns including a first set of patterns representing a calming heartbeat during soothing routines and a second set of patterns representing active engagement during conversational or play interactions, and to transition between the first and second sets of patterns based on detected changes in the primary user's emotional state.

12. The apparatus of claim 1, wherein the removable electronic module is configured to operate according to a multi-layer connectivity architecture comprising:
a primary layer in which core interaction features execute locally using the persistent emotional profile without a network connection;
a secondary layer in which the removable electronic module communicates via a short-range wireless protocol with a companion application to exchange configuration data, content, and interaction summaries; and
a tertiary layer in which the companion application communicates with remote services to perform artificial-intelligence processing and encrypted backup and restore of at least a portion of the persistent emotional profile while the removable electronic module lacks direct wide-area network connectivity.

13. The apparatus of claim 12, wherein, in the tertiary layer, the companion application is configured to transmit interaction summaries or audio streams derived from user interactions to a cloud-based artificial-intelligence service, receive generated responses, and route the responses back to the removable electronic module for playback through the audio output element, with the companion application enforcing privacy policies for the primary user.

14. The apparatus of claim 12, wherein the companion application is configured to maintain an encrypted cloud backup of at least the persistent emotional profile and, responsive to installation of a replacement removable electronic module, restore the persistent emotional profile from the encrypted cloud backup to the replacement removable electronic module.

15. The apparatus of claim 1, further comprising an age-tiered privacy management subsystem configured to associate the persistent emotional profile with an age classification of the primary user, select a privacy ruleset based on the age classification, and automatically enforce, for each privacy ruleset, limits on at least monitoring visibility, data collection scope, transcript retention, and third-party data sharing while the persistent emotional profile remains associated with the primary user across age classifications.

16. The apparatus of claim 15, wherein the age-tiered privacy management subsystem is further configured, upon detecting a transition of the primary user between age classifications, to preserve the persistent emotional profile and modify which caregivers or gatekeepers are permitted to access mood summaries, interaction histories, or other portions of the persistent emotional profile without deleting accumulated emotional data.

17. A removable electronic module for an interactive companion apparatus, the removable electronic module comprising:
a housing configured to be received in a docking interface of each of a plurality of companion device housings;
a processor; non-volatile memory storing a persistent emotional profile comprising personalized interaction data for a primary user; and
wireless communication circuitry;
wherein the processor is configured to execute emotional support routines using the persistent emotional profile, and the removable electronic module is configured, when moved between different ones of the plurality of companion device housings, to provide consistent emotional responses based on the persistent emotional profile independent of any specific companion device housing.

18. The removable electronic module of claim 17, further comprising a power subsystem including a rechargeable energy storage device and at least one of an inductive charging interface or a wired charging connector, and wherein the wireless communication circuitry comprises a short-range radio configured for communication with a companion application.

19. A method of providing persistent emotional support across multiple interactive companion device housings, the method comprising:
receiving, by a removable electronic module docked in a first companion device housing, interaction input from a primary user;
updating a persistent emotional profile stored in non-volatile memory of the removable electronic module based on the interaction input;
removing the removable electronic module from the first companion device housing and inserting the removable electronic module into a second companion device housing; and
personalizing interactions with the primary user in the second companion device housing based on the persistent emotional profile without retraining the removable electronic module.

20. The method of claim 19, further comprising, while the removable electronic module is docked in at least one of the first or second companion device housings, generating coordinated light and haptic patterns based on the persistent emotional profile, synchronizing at least a portion of the persistent emotional profile with a companion application via a short-range wireless link, and, responsive to a loss or damage event, restoring the persistent emotional profile from an encrypted cloud backup to a replacement removable electronic module.
